(19) United States
(12) Patent Application Publication
(10) Pub. No.: US 2015/0112282 A1
(43) Pub. Date: Apr. 23, 2015

(54) HOLLOW MICRONEEDLE AND SUBRETINAL SYRINGE FOR SUBRETINAL INJECTION OR EXTRACTION

(71) Applicant: INDUSTRY-ACADEMIC COOPERATION FOUNDATION, YONSEI UNIVERSITY, Seoul (KR)

(72) Inventors: Hyung Il Jung, Seoul (KR); Chang Yeol Lee, Gyeonggi-do (KR); Kwang Lee, Seoul (KR); Sung Ho Lee, Seoul (KR); Yong Sung You, Gyeonggi-do (KR)

(21) Appl. No.: 14/391,389

(22) PCT Filed: Apr. 9, 2013
(86) PCT No.: PCT/KR2013/002971

§ 371 Date: Oct. 8, 2014

(30) Foreign Application Priority Data
Apr. 9, 2012 (KR) 10-2012-0036554

(51) Int. Cl. A61F 9/00; A61M 5/158; A61M 5/14

(52) U.S. Cl. CPC A61F 9/0017; A61M 5/14; A61M 5/158
USPC 604/272; 427/2.28

(57) ABSTRACT

The present invention relates to a method for manufacturing a hollow microneedle for subretinal injection or extraction. The microneedle for subretinal injection or extraction is made of metal, thereby providing the strength and force for penetrating the sclera and retina. Due to the length and the angle of curvature, the microneedle for subretinal injection or extraction of the present invention can reach a suitable subretinal site, such as the midperiphery at which optic nerves are accumulated, and thus the present invention is effective for delivering a drug and can enhance the effectiveness of the drug.

### CROSS REFERENCE TO RELATED APPLICATIONS AND CLAIM OF PRIORITY

This patent application claims benefit under 35 U.S.C. 119(e), 120, 121, or 365(c), and is a National Stage entry from International Application No. PCT/KR2013/002971, filed Apr. 9, 2013, which claims priority to Korean Patent Application No. 10-2012-0036554, filed Apr. 9, 2012, entire contents of which are incorporated herein by reference.

### BACKGROUND

1. Technical Field

The present invention was made with the support of the Ministry of Health and Welfare, Republic of Korea, under Project No. A102003, which was conducted in the project titled “Drug customized for ocular diseases employing ultra-high-aspect microneedle technology” in the project named “Future fusion medical device development/treatment devices of medical R&D project” by the Industry-Academic Cooperation Foundation, Yonsei University under management of the Health Industry Development Institute, during the period of May 1, 2010 to Mar. 31, 2015.

This application claims priority to and the benefit of Korean Patent Application No. 10-2012-0036554 filed in the Korean Intellectual Property Office on Apr. 9, 2012, the entire contents of which are incorporated herein by reference.

The present invention relates to a method for fabricating a hollow microneedle for subretinal injection or extraction and a subretinal syringe or extractor.

2. Background Art

The macula lutea is a yellow spot at the center of the retina on which images of objects are focused and most visual cells are concentrated. Macular lesions are serious causes of visual impairments including loss of eyesight. Macular degeneration diseases are classified into an exudative form and a non-exudative form. The exudative form requires aggressive therapy. In current, laser coagulation, photodynamic therapy, antibody injection, vitrectomy, and the like are performed when boundaries of the macular degeneration are clearly defined, but there has been no complete treatment so far.

Recently, disclosed is a treatment of exudative macular degeneration using a subretinal syringe (retinal cannula from MedOne Inc.) in which a 40-gauge polymer tube is connected to a tip of a 23- or 25-gauge needle (see, FIG. 1). However, this subretinal syringe has faults in that the needle may not precisely penetrate a site to be injected due to flexible property of plastic polymer, causing retinal damage, and the bonding part with the needle may be prone to separation, causing medical accidents.

Meanwhile, Korean Patent Registration No. 0431454 discloses a subretinal syringe including: a guide having a guide groove formed therein, the guide groove having a larger diameter than a needle; a guide body coupled with the rear end of the guide and having a movement route formed therein; and a driver for allowing the needle to move forwardly or backwardly on the guide groove inside the guide body.

U.S. Pat. No. 5,409,457 discloses that the needle tip is bent perpendicularly in advance, and then allowed to penetrate the sclera vertically while a part of the sclera is pushed.

Throughout the entire specification, many papers and patent documents are referenced and their citations are represented. The disclosures of cited papers and patent documents are entirely incorporated by reference into the present specification, and the level of the technical field within which the present invention falls and details of the present invention are explained more clearly.

### SUMMARY

The present inventors endeavored to solve problems of conventional subretinal syringes, such as, especially, causing retinal damage; imprecisely penetrating by being bent due to low strength; being prone to separation of the bonding part with the needle, and the like. As a result, the present inventors fabricated a hollow microneedle optimized to a subretinal syringe by using hollow microneedle technology and confirmed that this hollow microneedle for subretinal injection can solve the above-mentioned problems, and thus completed the present invention.

Therefore, the present invention has been made in view of the above-mentioned problems, and an aspect of the present invention is to provide a method for fabricating a hollow microneedle for subretinal injection or extraction.

Another aspect of the present invention is to provide a subretinal syringe or extractor.

Other purposes and advantages of the present disclosure will become clarified by the following detailed description of invention, claims, and drawings.

In accordance with an aspect of the present invention, there is provided a method for fabricating a hollow microneedle for subretinal injection or extraction, the method including: (a) coating a solution of a viscous material onto a surface of a substrate; (b) placing a frame into contact with the solution of the viscous material; (c) preparing a solid microneedle by lifting the substrate, the frame, or both the substrate and the frame, so as to space apart the contacted frame and substrate from each other; (d) imparting a curved shape to the solid microneedle during step (c) or after completing step (c); (e) depositing a metal onto the solid microneedle with the curved shape; (f) plating the metal-deposited microneedle with a metal; (g) bevel-cutting the tip of the metal-plated solid microneedle; and (h) removing the solid microneedle to obtain a hollow microneedle with a curved shape.

The present inventors endeavored to solve problems of the conventional subretinal syringe, such as, especially, causing retinal damage; imprecisely penetrating by being bent due to low strength; being prone to separation of the bonding part with the needle, and the like. As a result, the present inventors fabricated a hollow microneedle optimized to a subretinal syringe by using hollow microneedle technology and confirmed that this hollow microneedle for subretinal injection can solve the above-mentioned problems.

### BRIEF DESCRIPTION OF THE DRAWINGS

FIG. 1 is an image of a subretinal syringe (retinal cannula from MedOne) of the related art.

FIG. 2a is a schematic diagram showing a subretinal syringe including a hollow microstructure of the present invention. 1: hollow microneedle, 2: syringe needle, 3. syringe connector

FIG. 2b shows another embodiment of the subretinal syringe including the hollow microstructure of the present invention. A short curve is formed at the end of the hollow microneedle.

FIG. 3 is a schematic view showing a curved portion in a hollow microneedle for subretinal injection or extraction of the present invention. “A” represents the maximum curved point. Two points, each of which is located at ½ the length of the microneedle in either direction from the maximum curved point, are designated by B′ and B″.

FIG. 4 is a schematic view of a hollow microneedle of the present invention. The arrow indicates the tip of the microneedle to which a bevel angle is imparted.

FIG. 5 is an image of a hollow microneedle for subretinal injection or extraction, fabricated in Example 1.

FIG. 6 is an image showing subretinal syringes including hollow microneedles for subretinal injection or extraction, which were fabricated in examples of the present invention.

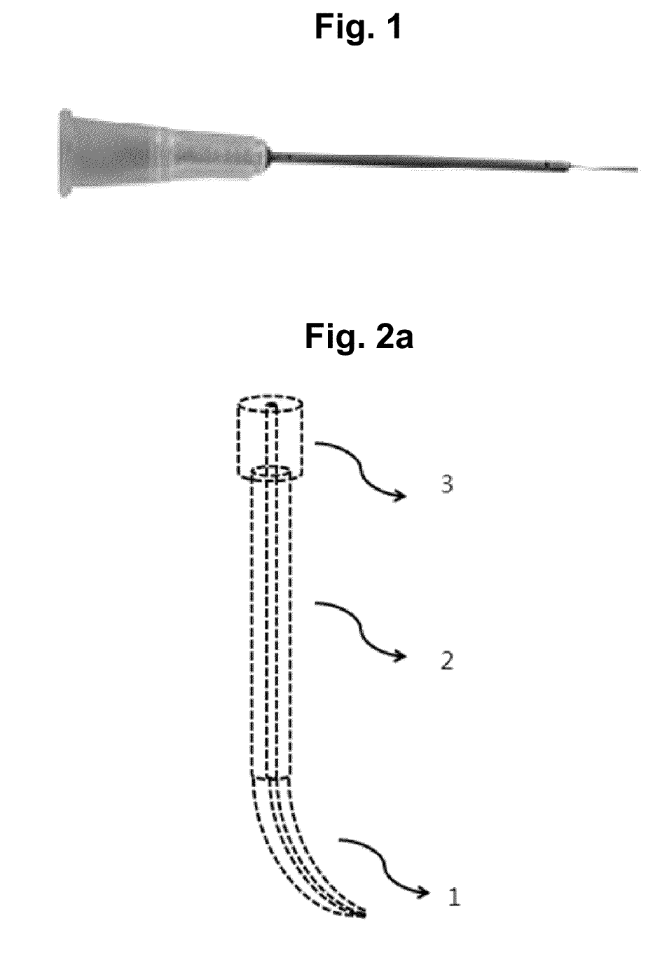

### DETAILED DESCRIPTION

The method of the present invention will be described by steps in detail as follows.

Step (a): Coating solution of viscous material onto surface of substrate

According to the method of the present invention, in order to prepare a solid microneedle, which is a mold for a hollow microneedle, first, a solution of a viscous material is coated onto a surface of a substrate.

The viscous material is used to prepare the solid microneedle, which is a mold for the hollow microneedle. As used herein, the term “viscous material” refers to a material, which is in a low-viscosity fluid state at a predetermined temperature or higher but has high viscosity when the temperature thereof is lowered to approximate the glass transition temperature thereof. Examples of the viscous material used in the present invention may include acryl based polymers, amide based polymers, acetyl based polymers, vinyl based polymers, epoxy based polymers, silicon based polymers, sulfone resins, polycarbonate based polymers, and copolymers thereof. However, without the limitation thereto, any viscous material that can be generally used in the art may be used.

Preferably, the viscous material used in the present invention has viscosity when being fluidized. The viscosity may be variously changed according to kinds, concentrations, and temperatures of viscous materials, organic solvents, and the like, and may be appropriately controlled to meet the purposes of the present invention. More preferably, the viscous material of the present invention shows a viscosity of 200000 cSt (centistoke) when being fluidized.

The fluidization of the viscous material may be performed through various methods known in the art. For example, a liquid polymer type of viscous material requires no fluidizing process, and a thermoplastic type viscous material has viscosity by being heated at a temperature higher than the melting point thereof and then being cooled to approximately the glass transition temperature thereof. Alternatively, a polymeric material may be fluidized by being dissolved in an appropriate organic solvent (e.g., anhydrous or hydrous lower alcohols having 1 to 4 carbon atoms, acetone, ethyl acetate, chloroform, 1,3-butylene glycol, hexane, diethyl ether, butyl acetate, etc.).

As used herein, the term “coating” refers to the formation of a layer of a certain material with a predetermined thickness on a target surface. A substrate providing the surface may be made of a polymeric material, an organic material, a metallic material, a ceramic material, a semiconductor material, or the like.

According to a preferable embodiment of the present invention, the coating thickness of the viscous material may be controlled within the range of 10 to 500 μm, more preferably 50 to 200 μm, and most preferably 75 to 165 μm.

According to a preferable embodiment of the present invention, the viscous material in step (a) is a high-molecular compound which is removable by an organic solvent.

As used herein, the term “high-molecular compound which is removable by an organic solvent” refers to a natural or synthetic compound having a molecular weight of 5,000 or more and having solubility to the organic solvent.

The high-molecular compound used in the present invention needs to be easily removed after metal depositing and metal plating for fabricating a hollow microneedle. The present inventors removed a high-molecular compound, which is one component of the metal-plated solid microstructure, by dissolving the high-molecular compound in an organic solvent.

The high-molecular compound used in the present invention is, more preferably, acrylonitrile styrene (AS), polyamide, polyethylene, polyester, polyacryl, polyacetyl, stylon, Teflon, polyvinylchloride, polyurethane, nylon, sulfone resin, or epoxy polymer. Most preferably, the high-molecular compound of the present invention is epoxy polymer.

The organic solvent used in the present invention includes, preferably, benzene, toluene, xylene (xylene), hexane, ether, acetone, alcohol, and amine. However, without the limitation thereto, any polar or non-polar solvent that can be generally used for dissolving a corresponding high-molecular compound may be used. For example, N-methyl pyrrolidine (NMP) may be used as a solvent when the epoxy polymer is used as a high-molecular compound.

Step (b): Placing frame into contact with solution of viscous material

After coating with the fluidized viscous material, preferably, the high-molecular compound, a lifting frame is placed into contact with the viscous material coat. According to a preferable embodiment of the present invention, the diameter of the lifting frame used herein is variable, and may be controlled within the range of preferably 1 to 1,000 μm, and most preferably 10 to 500 μm.

Preferable examples of the lifting frame used in the present invention include a cannula type stainless frame and a tube type frame having a passage.

According to a preferred embodiment of the present invention, the frame used in the present invention is a syringe needle. For example, the hollow microneedle is formed on a syringe needle of a syringe, which is composed of a syringe pump and the syringe needle, thereby providing a very efficient subretinal syringe.

More preferably, the frame used in the present invention is a syringe needle with a syringe connector mounted thereon.

As such, the microneedle is attached to and formed integrally with the syringe needle to provide a subretinal syringe.

The syringe needle as the frame is preferably 23-gauge or larger, more preferably 23- to 24-gauge, still more preferably 23- to 30-gauge or 23- to 27-gauge needle.

Step (c): Preparing solid microneedle by lifting substrate, frame, or both substrate and frame, so as to space apart contacted frame and substrate from each other

As used herein, the term “solid microneedle” refers to an integrally formed microneedle without a hollow part, as a template for a hollow microneedle.

In the present invention, the viscosity of the fluidized viscous material (preferably a high-molecular material) is increased while the temperature thereof is lowered to approximate the glass transition temperature thereof.

According to a preferable embodiment of the present invention, in order to provide a hollow microneedle suitable for subretinal injection, a solid structure is prepared by placing a hollow lifting frame into complete contact with the fluidized (for example, heated fluid type) viscous material, followed by upward lifting at a fast speed of about 3000 to 5000 μm/s. The prepared solid structure is rapidly vitrificated at room temperature.

The viscosity in step (c) influences various configurational factors of the finally fabricated hollow microneedle, that is, effective length, inner diameter, outer diameter, sharpness, aspect ratio, and the like of the finally fabricated hollow microneedle, and is a big variable in changing, especially, effective lengths of the solid microneedle and the hollow microneedle. In step (c), the higher the viscosity of the viscous material, the longer the effective length of the hollow microneedle.

According to a preferable embodiment of the present invention, the viscosity of the viscous material is controlled by adjusting the temperature of the viscous material within the range of higher than the glass transition temperature (T_{g}) but lower than 130° C.

As used herein, the term “glass transition temperature” refers to a temperature at which a fluid type of viscous material is solidified. Therefore, the solid microneedle cannot be prepared at a temperature lower than the glass transition temperature since the lifting process of the already solidified material is impossible. Also, the solid microneedle cannot be fabricated at a too high temperature since the lifting process is also impossible due to low viscosity thereof.

According to a preferable embodiment of the present invention, the viscosity of the viscous material, which is controlled in step (c), is 50 to 10,000 poise, more preferably to 8,000 poise, and still more preferably 100 to 6,500 poise.

As used herein, the term “space apart” refers to increasing the distance between the contacted substrates to thereby widen the space therebetween. The present inventors prepared the solid microneedle by lifting (upward moving) the frame in contact with the viscous material. However, the frame and the substrate may be spaced apart from each other by downwardly moving the substrate while the frame is fixed or upwardly moving and downwardly moving the frame and the substrate at the same time.

According to the present invention, the lifting speed of the viscous polymer is controlled, so that various configurational factors of the finally fabricated hollow microneedle, that is, effective length, inner diameter, outer diameter, sharpness, aspect ratio, and the like, can be controlled and, especially, effective lengths of the solid microneedle and the hollow microneedle can be controlled.

As used herein, the term “lifting speed” refers to the inclusion of a relative speed of the frame and the substrate which move away from each other when the frame and the substrate move upwardly and downwardly at the same time, as well as an upward or downward moving speed of the frame or the substrate.

The lifting speed used in the present invention is 0.1 to 2,000 μm/s, and most preferably 1 to 1,000 μm/s. The length of the final solid microneedle can be controlled by the correlation (product) between the lifting speed and the lifting time.

Step (d): Imparting curved shape to solid microneedle

During step (c) or after completing step (c), a curved shape is imparted to the solid microneedle to obtain a solid microneedle having a curved portion.

For example, the imparting of the curved shape may be performed by applying force (e.g., wind power, hot wind power) to a predetermined portion of the solid microneedle to bend the microneedle, before the solid microneedle is completely solidified after the lifting procedure for preparing the solid microneedle.

Alternatively, the curved portion of the solid microneedle may be formed by bending the solid microneedle through application of hot wind to a predetermined portion of the solid microneedle after the solid microneedle is solidified after the lifting procedure for preparing the solid microneedle.

According to a preferable embodiment of the present invention, the imparting of the curved shape may be performed by using wind power and more preferably hot wind.

The imparting of the curved shape may be performed such that the hollow microneedle preferably has a curved angle of 10 to 70 degrees. As used herein, the term “curved angle” refers to an angle from the arctangent (tan⁻¹) of the value obtained by dividing the difference in y coordinates by the difference in x coordinates of specific two positions in the portion of the microneedle to which the curved shape is imparted, that is, the curved portion of the microneedle: tan⁻⁻¹ [(y₁ coordinate−y₂ coordinate)/(x₁ coordinate−x₂ coordinate)]. According to the present invention, the base part of the microneedle is not bent but has a linear shape while the curved shape is imparted to the middle portion of the microneedle. Therefore, as used herein, the term “curved angle” value is calculated by using coordinate values of two specific positions in the curved portion, which is formed at the middle region of the microneedle of the present invention.

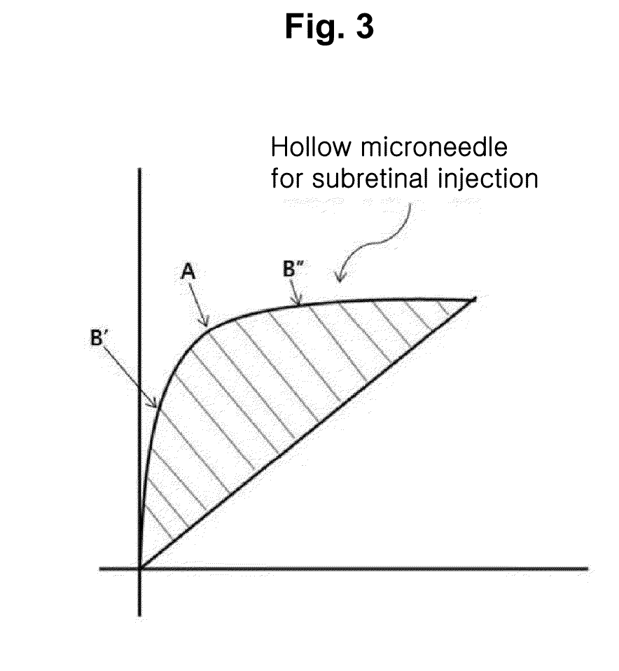

As used herein, the term “curved portion” refers to a portion of the microneedle, which is between two points (B′ and B″ in FIG. 3) each of which is located at ½ the length of the microneedle in either direction from a point at which the curvature is maximum, that is, the maximum curved point (“A” in FIG. 3).

If the curved angle of the microneedle is smaller than 10 degrees, such a small curved degree is inefficient in reaching the subretinal region (e.g., subretinal region near the optical nerve) at which the hollow microneedle for subretinal injection or extraction of the present invention is to arrive. If the curved angle of the microneedle is larger than 70 degrees, such a large curved degree is inefficient in reaching a subretinal region at which the hollow microneedle for subretinal injection or extraction is to arrive, and besides, the force of the microneedle to penetrate the sclera and the retina is significantly reduced.

More preferably, the curved angle of the microneedle fabricated in the present invention is 40 to 60 degrees.

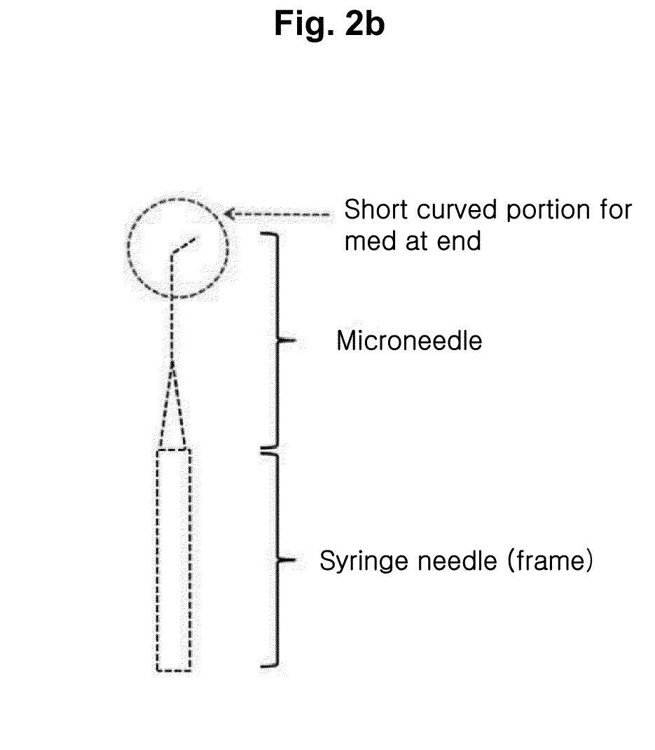

Embodiments of the subretinal syringe including the hollow microstructure of the present invention are shown in FIGS. 2a and 2b. The curved portion may be introduced at the middle portion of the hollow microneedle as shown in FIG. 2a. Alternatively, a short curved portion may be introduced at the end of the hollow microneedle as shown in FIG. 2b. In addition, the curved portion may have a curved shape as shown in FIG. 2a, or a linear shape as shown in FIG. 2b. That is, the curved shape in the present invention is preferably construed to include any shape that can be imparted to the hollow microneedle to change the direction of the hollow microneedle.

Step (e): Depositing metal onto solid microneedle with curved shape

According to the present invention, a metal is deposited onto the solid microneedle with the curved shape, so as to promote a subsequent metal plating reaction for fabricating the hollow microneedle.

As used herein, the term “deposition” refers to the process by which, in order to enhance mechanical strength of a substrate, a coating material is physically or chemically evaporated or sublimated into atomic or molecular level forms, which are then condensed on a surface of the substrate, thereby forming a film on the surface of the substrate. The deposition in the present invention may be performed by any physical vapor deposition or chemical vapor deposition that can be generally employed in the art.

According to a preferable embodiment of the present invention, the metal for the depositing is stainless steel, aluminum (Al), chrome (Cr), nickel (Ni), gold (Au), silver (Ag), copper (Cu), titanium (Ti), cobalt (Co), or an alloy thereof. More preferably, silver (Ag) is chemically deposited by using the Tollens reaction.

According to an example of the present invention, silver (Ag), which is precipitated through a reduction reaction using the Tollens' reagent (Ag₂O+NH₄OH+H₂O), was deposited on the solid microneedle. The Tollens reaction is more favorable in the deposition of metal on the target substrate, as compared with physical vapor deposition, since it requires no heating, pressurizing, and separate cooling.

Step (f): Plating surface of metal-deposited solid microstructure with metal

The metal-deposited solid microneedle is plated with a metal, thereby provide a base of a hollow microneedle.

One characteristic of the present invention is to perform metal plating without masking the tip of the microneedle after the metal depositing. The conventional technologies of fabricating a hollow microneedle (e.g., Koran Patent Registration No. 781702 and Korean Patent Application No. 10-2010-00066940) necessarily entail a procedure of masking the tip of the microneedle before metal plating. According to the present invention, the fabricating time is shortened and the fabricating convenience is improved without performing the above-mentioned procedure.

In order to fabricate a hollow microneedle for subretinal injection or extraction, the conventional technology follows the procedure of “coating-lifting-metal depositing-tip masking-metal plating-solid structure removing-bevel cutting” but the present invention follows the procedure of “coating-lifting-metal depositing-metal plating-solid structure removing-bevel cutting”.

The metal plating thickness in the present invention is 5 to 100 μm, and more preferably 10 to 50 μm.

The material for plating used in the present invention includes, for example, nickel, stainless steel, aluminum, chrome, cobalt based alloys, titanium, and an alloy thereof. However, without the limitation thereto, any metal that is known to be biocompatible; have no toxicity or carcinogenicity; have no body rejection; retain good mechanical properties, such as tensile strength, elongation, and wear resistance; and have corrosion resistance to endure under the corrosive environment of the human body in the art may be used.

According to a preferable embodiment of the present invention, the metal for the plating is stainless steel, aluminum (Al), chrome (Cr), nickel (Ni), gold (Au), silver (Ag), copper (Cu), titanium (Ti), cobalt (Co), or an alloy thereof. More preferably, the metal for the plating of the present invention is nickel (Ni).

Step (g): Bevel-cutting metal-plated solid microneedle

Then, the tip of the metal-plated solid microneedle is bevel-cut. The bevel angle, which is imparted by the bevel cutting, is preferably 5 to 50 degrees.

According to a preferable embodiment of the present invention, the bevel angle is formed at an inside of the microneedle with a curved shape.

The bevel angle of the conventional hollow microneedle is 30 to 90 degrees, but the microneedle customized for ocular diseases of the present invention has a bevel angle similar to that of the general syringe.

The bevel cutting may be performed by any deliberate cutting method that can be generally employed in the art. Laser or a microsaw (dicing saws) may be used for bevel cutting, and more preferably laser cutting may be performed. The bevel angle may be adjusted to provide sharpness suitable for subretinal injection.

As for the hollow microneedle for subretinal injection or extraction, the bevel angle is 5 to 50 degrees, more preferably 5 to 20 degrees, still more preferably 5 to 15 degrees, and still more preferably 10 to 15 degrees.

Step (h): Removing solid microstructure to obtain a hollow microstructure with a curved shape

The solid microneedle is removed to obtain a hollow microneedle with a curved shape. The solid microneedle may be removed by being dissolved in an appropriate organic solvent, may be burned, or may be physically removed. Preferably, the solid microneedle may be removed by using above-listed appropriate organic solvents.

The hollow microneedle for subretinal injection or extraction, which is fabricated through the above-described procedure, has structural and physical characteristics for penetrating the sclera and the retina and injecting a drug into a suitable site under the retina.

According to a preferable embodiment of the present invention, the effective length of the finally fabricated hollow microneedle is 1 to 10 mm, more preferably 5 to 10 mm, and still more preferably 8 to 10 mm.

The length of the microneedle that has been developed until now is only up to 2 mm. The microneedle of the present invention overcomes this limit value and provides an effective length suitable for subretinal injection. The microneedle of the present invention has such a length that the microneedle can penetrate the sclera and the retina to inject a drug into a site under the retina. Here, the sclera and the retina have the highest strength among outer walls of the eyeball.

As used herein, the term “effective length” refers to a length from the upper end of the microneedle to a surface of the substrate at the lower end thereof, that is, a vertical length of the needle frame. As used herein, the term “aspect ratio” refers to a ratio of the vertical length from the upper end of the microneedle to the surface of the substrate at the lower end of the microneedle, that is, the needle frame over the maximum diameter of the microneedle (height to diameter at base).

According to a preferable embodiment of the present invention, the inner diameter at the upper end of the finally fabricated hollow microneedle is 20 to 150 μm, and more preferably 40 to 150 μm.

As used herein, the term “upper end” refers to one end of the microneedle, which has a minimum diameter in the microneedle, and the term “lower end” refers to the bottom end of the microneedle, which is in contact with the substrate (frame).

According to another aspect of the present invention, provided is a subretinal syringe or extractor including a hollow microneedle having an effective length of 1 to 10 mm, an inner diameter at the upper end of 20 to 150 μm, a bevel angle of 5 to 50 degrees, and a curved angle of 10 to 70 degrees.

According to a preferable embodiment of the present invention, the subretinal syringe of the present invention has a structure in which a syringe needle is mounted on a base portion of the hollow microneedle, the syringe needle being openly connected to the hollow microneedle.

More preferably, the subretinal syringe of the present invention includes a syringe connector, a syringe needle, and a hollow microneedle (see, FIG. 2). The syringe connector is a connection portion between a syringe pump and the syringe needle. The syringe needle is connected between the syringe pump and the hollow microneedle while a fluid flows therethrough. The hollow microneedle is connected to the end of the syringe needle while a fluid flows therethrough.

According to a preferable embodiment of the present invention, the curved angle of the hollow microneedle is 40 to 60 degrees.

According to a preferable embodiment of the present invention, the hollow microneedle of the present invention is made of stainless steel, aluminum (Al), chrome (Cr), nickel (Ni), gold (Au), silver (Ag), copper (Cu), titanium (Ti), cobalt (Co), or an alloy thereof.

According to a preferable embodiment of the present invention, the bevel angle of the hollow microneedle is 5 to 15 degrees, and more preferably 10 to 15 degrees.

According to a preferable embodiment of the present invention, the effective length of the hollow microneedle is 5 to 10 nm.

According to a preferable embodiment of the present invention, the inner diameter at the upper end of the hollow microneedle is 50 to 100 μm.

According to a preferable embodiment of the present invention, the hollow microneedle is fabricated by the above-described method of the present invention.

According to a preferable embodiment of the present invention, the hollow microstructure has a strength of 0.1 to 5.0 N, more preferably 0.1 to 2.0 N, still more preferably 0.5 to 2.0 N, and still more preferably 1.0 to 2.0 N.

The minimum force necessary for penetrating the sclera of the eyeball is known to be 1 N or smaller (J. S. Pulido et al., Scleral penetration force requirements for commonly used intravitreal needles, EYE, 21:1210-1211(2007)). Therefore, the strength of the hollow microneedle of the present invention is sufficient to easily penetrate the sclera and the retina to deliver a drug to a site under the retina.

According to a preferable embodiment of the present invention, the use of the hollow microneedle is to penetrate the sclera and the retina to inject a drug into a suitable site (e.g., the subretinal center on that optical cells or optical nerves are concentrated) under the retina.

According to a preferable embodiment of the present invention, the microneedle of the present invention penetrates the sclera to extract (i.e., remove) subretinal materials, thereby treating retinal detachment. Therefore, the use of the hollow microneedle of the present invention can lead to effective treatment of retinal detachment. Retinal detachment is a disease of the eye in which the retina in charge of the eyesight is detached from the choroid by holes, tears, dialysis, and the like. Here, a vitreous material penetrates into a space between the retina and the choroid, resulting in further detachment of the retina. Therefore, the early treatment of the retinal detachment is essential. Since chronic retinal detachment is untreatable, the retina is allowed to attach to the choroid as soon as possible for nutrient supply. As for the treatment of retinal detachment, it is general to attach the retina to the choroid, thereby repairing the detachment. For retinal attachment, Scleral buckling (Schwartz S G, Flynn H W., Curr Opin Ophthalmol. 2006; 17:245-250), pars plana vitrectomy (PPV, Schwartz S G, Flynn H W., Clin Ophthalmol., 2008; 2:57-63), gas injection (Itakura H, Kishi S. Graefes Arch Clin Exp Ophthalmol. 2009; 247 (8):1147-50), silicon oil injection (Moisseiev J et. al., Retina. 1998; 18 (3):221-7), intraocular laser surgery, and the like have been used. However, preferred methods for treating retinal detachment are controversial until now.

The hollow microneedle of the present invention effectively removes materials between the retina and the choroid while minimizing retinal damage due to its distinct structure, thereby attaining safe treatment of retinal detachment.

Features and advantages of the present invention are summarized as follows:

(i) The hollow microneedle for subretinal injection or extraction of the present invention can have a strength or force to penetrate the sclera and the retina since it is made of a metal.

(ii) The hollow microneedle for subretinal injection or extraction of the present invention can minimize retinal damage since it has a small diameter.

(iii) The hollow microneedle for subretinal injection or extraction of the present invention has an improved length and an improved curved angle, so that the hollow microneedle can reach an appropriate site under the retina, for example, the retinal center on which optical nerves are concentrated. Therefore, the hollow microneedle is effective in drug delivery and enhances drug effects.

Further, the hollow microneedle for subretinal injection or extraction of the present invention is very useful in treating retinal detachment by extracting retinal materials.

(iv) The retinal syringe of the present invention is easily compatible with general syringes.

(v) There can be provided a subretinal syringe in which, when a syringe needle is used as the frame, the hollow microneedle is attached to and thus formed integrally with the syringe needle.

### Mode for Carrying Out the Invention

Hereinafter, the present invention will be described in detail with reference to examples. These examples are only for illustrating the present invention more specifically, and it will be apparent to those skilled in the art that the scope of the present invention is not limited by these examples.

### EXAMPLES

### Example 1

### Fabrication of Microneedle Device for Subretinal Injection or Extraction I

A solid microneedle was prepared by using SU-8 2050 photoresist (purchased from Microchem) having a viscosity of 14,000 cSt. The SU-8 2050 photoresist was coated on a cover glass of 1.5 cm×1.5 cm at 1000 RPM to maintain a thickness of about 160 μm. The cover glass was heated on a hot plate at 120° C. for about 30 minutes, to maintain the fluidity of the SU-8 2050 photoresist. Then, a 23-gauge syringe needle with a syringe connector, the syringe needle having a flat end, was placed in contact with the SU-8 2050, followed by vertical lifting, thereby preparing a solid structure having a diameter of 50 μm and a length of 5 to 10 mm. Then, hot wind was applied to the solid structure for several seconds to impart a curve of 45 to 60 degrees to the solid structure.

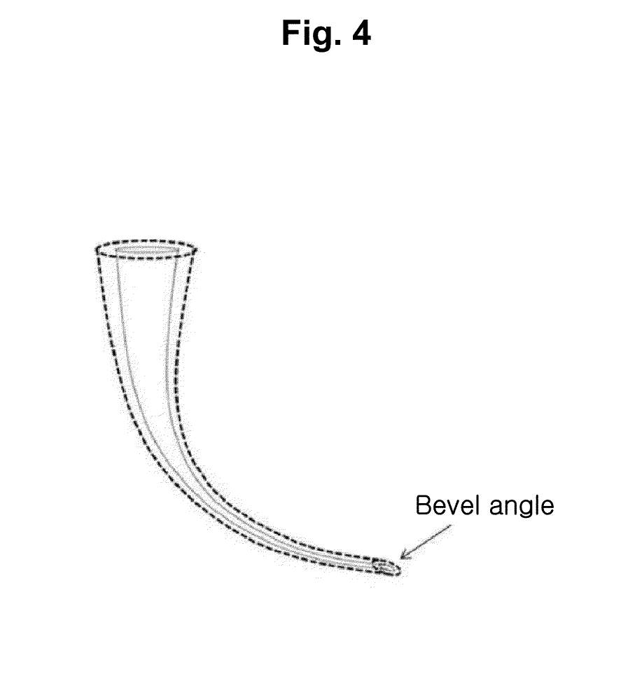

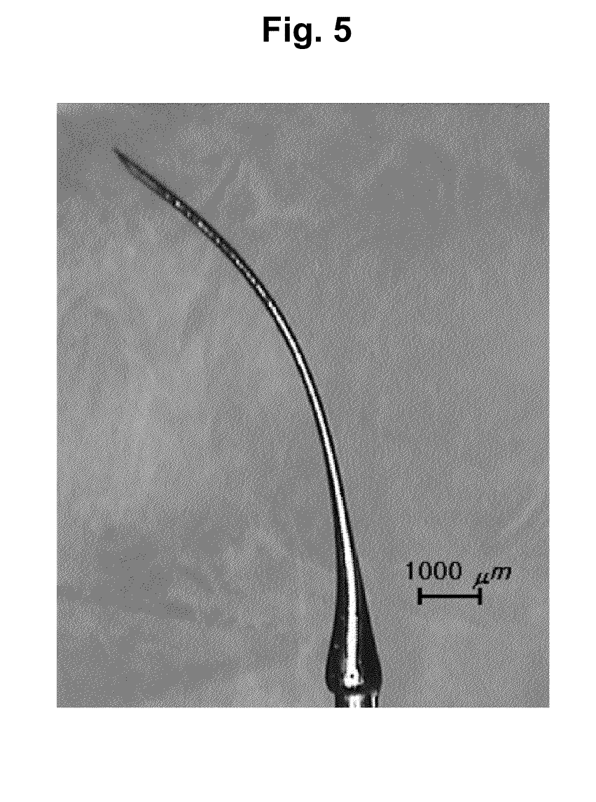

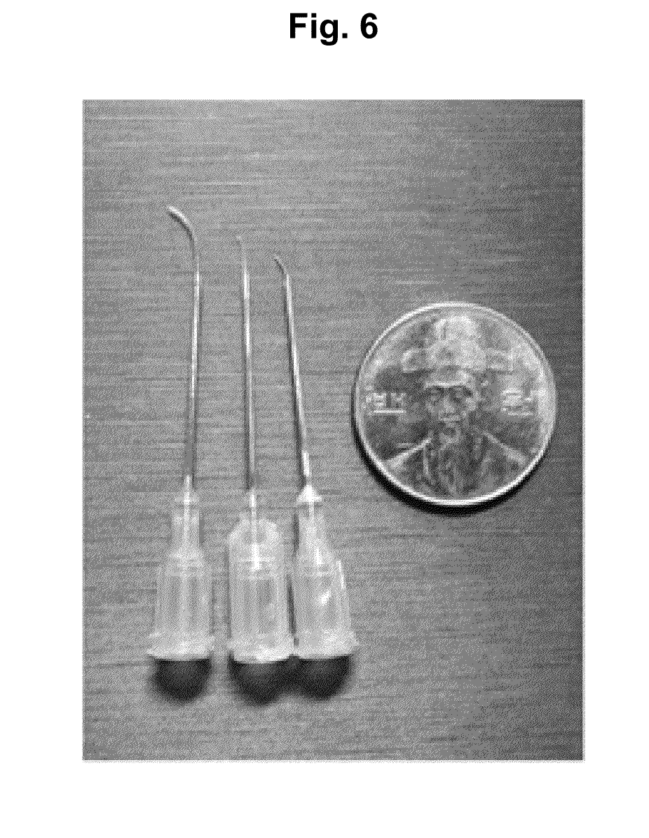

The resultant solid structure was subjected to silver plating by using the Tollens' reagent, and then subjected to nickel electroplating. The nickel electroplating was performed at 0.206 μm/min for 1 A/dm² for 75 minutes, so that the plating metal thickness was 20 μm. The end of the plated solid structure was cut by application of a bevel of 15 degrees, and then the solid structure was removed by the SU-8 remover (purchased from Microchem) or acetone, thereby fabricating a hollow microneedle type microneedle device for subretinal injection or extraction (FIG. 4).

The fabricated microneedle for subretinal injection or extraction is a hollow microneedle having an outer diameter at the upper end of 120 μm, an inner diameter at the upper end of 50 μm, a diameter at the lower end of 350 μm, and a length of 9.02 mm. The strength of the fabricated hollow microneedle exhibits 1 to 2 N, which is greater than the minimum strength to penetrate the retina.

### Example 2

### Fabrication of Microneedle Device for Subretinal Injection or Extraction II

A solid microneedle was prepared by using SU-8 2050 photoresist (purchased from Microchem) having a viscosity of 14,000 cSt. The SU-8 2050 photoresist was coated on a cover glass of 1.5 cm×1.5 cm at 1000 RPM to maintain a thickness of about 160 μm. The cover glass was heated on a hot plate at 120° C. for about 1 hour, to maintain the fluidity of the SU-8 2050 photoresist. Then, a 23-gauge syringe needle having a flat end was placed in contact with the SU-8 2050 photoresist, followed by vertical lifting, thereby preparing a solid structure having a diameter of 20 to 60 μm and a length of 5 to 10 mm. In this case, while the temperature of the cover glass, that is, the substrate is slowly lowered to 90° C. (polymer adhesive strength: 1 N, viscosity: 100 poise) and 60° C. (polymer adhesive strength: 2 N, viscosity: 6,500 poise), the lifting frame (syringe needle) was lifted at a speed of 10 μm/s for 5 minutes, respectively. The resultant solid structure was subjected to silver plating by using the Tollens' reagent, and then subjected to nickel electroplating. The nickel electroplating was performed at 0.206 μm/min for 1 A/dm² for 75 minutes, so that the plating metal thickness was 20 μm. The end of the plated solid structure was cut by application of a bevel of 15 degrees, and then the solid structure was removed by the SU-8 remover (purchased from Microchem) or acetone, thereby fabricating a hollow microneedle type microneedle device for subretinal injection or extraction.

The fabricated microneedle for subretinal injection or extraction is a hollow microneedle having an outer diameter at the upper end of 110 μm, an inner diameter at the upper end of 40 μm, a diameter at the lower end of 350 μm, and a length of 3 to 6 mm. The strength of the fabricated hollow microneedle exhibits 1 to 2 N, which is greater than the minimum strength to penetrate the retina.

### Example 3

### Fabrication of Microneedle Device for Subretinal Injection or Extraction III

A solid microneedle was prepared by using SU-8 2050 photoresist (purchased from Microchem) having a viscosity of 14,000 cSt. The SU-8 2050 photoresist was coated on a cover glass of 1.5 cm×1.5 cm at 1000 RPM to maintain a thickness of about 160 μm. The cover glass was heated on a hot plate at 120° C. for about 1 hour, to maintain the fluidity of the SU-8 2050 photoresist. Then, a 23-gauge syringe needle having a flat end was placed in contact with the SU-8 2050 photoresist, followed by vertical lifting, thereby preparing a solid structure having a diameter of 20 to 60 μm and a length of 5 to 10 mm. In this case, while the temperature of the cover glass, that is, the substrate is slowly lowered to 70 to 60° C., the lifting frame (syringe needle) was lifted at a speed of 5 μm/s and 10 μm/s for 5 minutes, respectively.

The resultant solid structure was subjected to silver plating by using the Tollens' reagent, and then subjected to nickel electroplating. The nickel electroplating was performed at 0.206 μm/min for 1 A/dm² for 75 minutes, so that the plating metal thickness was 20 μm. The end of the plated solid structure was cut by application of a bevel of 15 degrees, and then the solid structure was removed by the SU-8 remover (purchased from Microchem) or acetone, thereby fabricating a hollow microneedle type microneedle device for subretinal injection or extraction.

The fabricated microneedle for subretinal injection or extraction is a hollow microneedle having an outer diameter at the upper end of 120 μm, an inner diameter at the upper end of 50 μm, a diameter at the lower end of 350 μm, and a length of 5 to 8 mm. The strength of the fabricated hollow microneedle exhibits 1 to 2 N, which is greater than the minimum strength to penetrate the retina.

Microneedles for subretinal injection or extraction having various dimensions (inner diameter, diameter, and length) were fabricated by adjusting the polymer temperature and the lifting speed in the examples.

### Example 4

### Treatment of Retinal Detachment Using Microneedle for Subretinal Injection or Extraction

In order to verify whether the fabricated microneedle for subretinal injection or extraction has excellent workability, the retinal detachment treatment experiment was conducted by using the microneedle of Example 1 among the microneedles for subretinal injection or extraction fabricated as above.

A frozen porcine eye was thawed for 2 hours, and then fixed in the fixing frame. The porcine eye, which was unfrozen from the frozen state, had severe retinal detachment, and thus this was used for experiment. A water supply and a light source were connected to the porcine eye at the one o'clock direction and seven o'clock direction, respectively. Then, the vitreous body was removed, and the retina was perforated by using the hollow microneedle for subretinal injection or extraction of the present invention, followed by extraction. Before the retina was perforated, most of the retina was detached from the choroid to fill the vitreous cavity. The perforation was performed by using the hollow microneedle of the present invention and the extracting was performed by using the LEICA instrument at a pressure of 600 mmHg. It was confirmed that the retinal detachment was repaired and the retina and the choroid were attached to each other within several seconds after the extraction.

Therefore, it can be seen that the hollow microneedle of the present invention can minimize the retinal damage and effectively remove the materials between the retina and the choroid while being used for retinal injection or extraction.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for fabricating a hollow microneedle for subretinal injection or extraction, the method comprising:
(a) coating a solution of a viscous material onto a surface of a substrate;
(b) placing a frame into contact with the solution of the viscous material;
(c) preparing a solid microneedle by lifting the substrate, the frame, or both the substrate and the frame, so as to space apart the contacted frame and substrate from each other;
(d) imparting a curved shape to the solid microneedle during step (c) or after completing step (c);
(e) depositing a metal onto the solid microneedle with the curved shape;
(f) plating the metal-deposited microneedle with a metal;
(g) bevel-cutting the tip of the metal-plated solid microneedle; and
(h) removing the solid microneedle to obtain a hollow microneedle with a curved shape.

2. The method of claim 1, wherein the frame is a syringe needle.

3. The method of claim 2, wherein the syringe needle is a 23-gauge or larger syringe needle.

4. The method of claim 1, wherein the viscosity of the viscous material is controlled by adjusting the temperature of the viscous material within the range of higher than the glass transition temperature thereof but lower than 130° C.

5. The method of claim 1, wherein the metal for the plating is stainless steel, aluminum (Al), chrome (Cr), nickel (Ni), gold (Au), silver (Ag), copper (Cu), titanium (Ti), cobalt (Co), or an alloy thereof.

6. The method of claim 1, wherein the hollow microneedle with the curved shape has a curved angle of 10 to 70 degrees.

7. The method of claim 6, wherein the curved angle is 40 to 60 degrees.

8. The method of claim 1, wherein the bevel-cutting is performed such that the bevel angle at the tip of the hollow microneedle is 5 to 50 degrees.

9. The method of claim 8, wherein the bevel angle at the tip of the hollow microneedle is 5 to 15 degrees.

10. The method of claim 1, wherein the effective length of the hollow microneedle is 1 to 10 mm.

11. The method of claim 1, wherein the inner diameter at the upper end of the hollow microneedle is 20 to 150 μm.

12. The method of claim 1, wherein the frame is a syringe needle having a syringe connector.

13. A subretinal syringe or extractor comprising a hollow microneedle, wherein the hollow microneedle has an effective length of 1 to 10 mm, an inner diameter at the upper end of 20 to 150 μm, a bevel angle of 5 to 50 degrees, and a curved angle of 10 to 70 degrees.

14. The subretinal syringe or extractor of claim 13, wherein a syringe needle is mounted on a base part of the hollow microneedle, the syringe needle being openly connected to the hollow microneedle.

15. The subretinal syringe or extractor of claim 13, wherein the curved angle is 40 to 60 degrees.

16. A subretinal syringe or extractor comprising a hollow microneedle, wherein the hollow microneedle has an effective length of 1 to 10 mm, an inner diameter at the upper end of 20 to 150 μm, a bevel angle of 5 to 50 degrees, and a curved angle of 10 to 70 degrees, and the hollow microneedle is fabricated by the method of claim 1.

17. The subretinal syringe or extractor of claim 13, wherein the strength of the hollow microneedle is 0.1 to 5.0 N.

18. The subretinal syringe or extractor of claim 13, wherein the hollow microneedle delivers a drug subretinally.

19. The subretinal syringe or extractor of claim 13, wherein the hollow microneedle extracts or removes materials under the retina.
